(19) United States
(12) Patent Application Publication
(10) Pub. No.: US 2008/0071067 A1
(43) Pub. Date: Mar. 20, 2008

(54) Methods of purifying proteins from egg white

(75) Inventors: Liang M. Chen, Athens, GA (US); Markley C. Leavitt, Watkinsville, GA (US); Jeffrey C. Rapp, Athens, GA (US)

Correspondence Address: AVIGENICS, INC., 111 RIVERBEND ROAD, ATHENS, GA, 30605, US

(73) Assignee: AviGenics, Inc.

(21) Appl. No.: 11/901,029

(22) Filed: Sep. 14, 2007

Related U.S. Application Data
(60) Provisional application No. 60/846,183, filed on Sep. 20, 2006.

(51) Int. Cl. C07K 1/18

(52) U.S. Cl. 530/416

(57) ABSTRACT

The invention provides methods of purifying proteins, e.g., cytokines, from egg whites.

### RELATED APPLICATION INFORMATION

This application claims the benefit of priority to U.S. Provisional Application No. 60/846,183, filed Sep. 20, 2006, the disclosure of which is incorporated in its entirety herein by reference.

### BACKGROUND OF THE INVENTION

Transgenic avians, e.g., transgenic chickens, are a desirable expression system for obtaining proteins for use in pharmaceutical or other commercial applications that require large amounts of protein. A hen can lay up to 330 eggs per year, each containing 6.5 grams of protein. About 3.5 grams of the total protein is egg white, of which 90% is accounted for by seven genes; the ovalbumin gene alone accounts for 2 grams of egg white protein. Tubular gland cells line the lumen of the magnum of the oviduct and continually express and store egg white proteins in intracellular granules. As a yolk passes through the magnum, the granule contents are released and deposited onto the yolk. Advantages of protein production in chicken eggs include short generation times and prolific rates of reproduction via artificial insemination. Various proteins have been expressed in eggs of transgenic chickens. See, e.g., U.S. Pat. No. 6,730,822, issued May 4, 2004 and US patent publication No. 20060015960. The disclosures of this patent and this published patent application are incorporated in their entirety herein by reference.

Many cytokines, e.g., erythropoietin, granulocyte colony-stimulating factor (GC-SF), interferons, and granulocyte-macrophage colony-stimulating factor (GM-CSF), are of interest to the pharmaceutical industry. The cytokines can readily be obtained in significant quantities from transgenic chickens. Traditional methods of isolating cytokines, however, often rely on use of immunoaffinity procedures or other procedures suitable for small scale purification. For such a large-scale protein production, such a purification procedure is costly. The present invention is based on the discovery of an inexpensive and efficient method for purifying cytokines from egg whites.

### SUMMARY OF THE INVENTION

The invention provides methods of purifying heterologous proteins, e.g., cytokines, present in egg white. Typically, the methods include: acidifying an egg white preparation comprising the heterologous protein, e.g., cytokine; performing a first and a second ion exchange chromatography; and performing chromatography based on hydrophobic interaction principles, thereby purifying the heterologous protein. Cytokines that can be purified using this method include, without limitation, granulocyte colony-stimulating factor (G-CSF); interferons such as interferon-α or interferon-β; granulocyte-macrophage colony-stimulating factor (GM-CSF); and erythropoietin.

In certain embodiments, the first and the second ion exchange chromatography are cation exchange chromatography. Often, the second cation exchange chromatography is at a pH that is lower than the first cation exchange chromatograph. In one embodiment, the chromatography based on hydrophobic interaction principles is reverse phase chromatography. In another embodiment, the chromatography based on hydrophobic interaction principles is hydrophobic interaction chromatography.

The egg white preparation comprising the heterologous protein, e.g., cytokine, is acidified, for example, to a pH in the range of about 2.0 to about 6.5, e.g., to a pH of about 4.0 to about 5.5.

In one embodiment, the invention provides for methods of purifying a heterologous protein, e.g., a cytokine, from an egg white preparation comprising the protein, the method comprising: acidifying the egg white preparation to a pH of about 4.0 to about 6.5; performing a first cation exchange chromatography; performing a second cation exchange chromatography, wherein the second cation exchange chromatography is at a different pH, typically a lower pH, than the first cation exchange chromatography; and performing chromatography based on hydrophobic interaction; thereby purifying the heterologous protein from the egg white preparation. The protein that is purified using this method can be a cytokine, e.g., and without limitation, G-CSF; an interferon such as interferon-α or interferon-β; erythropoietin, or GM-CSF. In certain embodiments, the first cation exchange chromatography step is at a pH of about 5.0 and the second step is at a pH of about 4.0. Examples of chromatography based on hydrophobic interaction are reversed phase and hydrophobic interaction chromatography.

Any useful combination of features described herein is included within the scope of the present invention provided that the features included in any such combination are not mutually inconsistent as will be apparent from the context, this specification, and the knowledge of one of ordinary skill in the art. For example, the term transgenic can encompass the term transchromosomal and methodologies useful for transgenic animals (e.g., avians) and cells disclosed herein may also be employed for transchromosomal avians and avian cells.

Additional objects and aspects of the present invention will become more apparent upon review of the detailed description set forth below when taken in conjunction with the accompanying figures, which are briefly described as follows.

### DETAILED DESCRIPTION OF THE INVENTION

### Definitions

A “cytokine” in the context of this invention is a member of a group of protein signaling molecules that may participate in cell-cell communication in immune and inflammatory response. Cytokines are typically small, water-soluble glycoproteins that have a mass of about 8-35 kDa. Usually, the isoelectric point of cytokines that can be isolated or purified as disclosed herein, e.g., using cation exchange chromatography, is between about 6.0 and about 7.0, typically, about 6.5. Therefore, at pH 6.5 the cytokines will bind to a cation exchange material during the purification process. In addition, cytokines are often quite hydrophobic providing for use of hydrophobic interaction in the purification process.

A “heterologous” protein (e.g., a “heterologous” cytokine) as used herein refers to a protein that is non-naturally occurring in the producing organism. The protein, e.g., a cytokine may be expressed in a transgenic avian that contains a transgene in its genome that encodes the protein, wherein the protein is expressed in the oviduct and is deposited in egg white produced by the avian.

The term “purifying” as used herein refers to increasing the degree of purity of the protein in a preparation by removing one or more contaminants, e.g., naturally occurring avian egg protein contaminants such as albumen, from the preparation. “Removal” of the contaminant may not be a complete removal. A “purified” protein, e.g., a “purified cytokine, in accordance with the invention is typically at least 70%, often at least 80% by weight of a composition, and may be 90% or 99% or more by weight of a composition after purification.

A “cation exchange chromatography” refers to a separation employing a solid phase resin that is negatively charged, and thus can bind positively charged molecules in an aqueous solution passed over or through the solid phase. Examples of negatively charged molecules attached to the solid phase to form a cation exchange resin are carboxylate or sulfonate.

An “anion exchange chromatography” refers to a separation employing a solid phase resin that is positively charged, and thus can bind negatively charged molecules in an aqueous solution passed over or through the solid phase. Commonly used anion exchange resins are Q-resins (a quaternary amine resin) and DEAE resin.

The term “acidifying” as used in the context of this invention refers to reducing the pH of a mixture, solution, or preparation.

An “egg white preparation” as used herein refers to egg whites that have been separated from the yolk. It is understood that there may be residual yolk present in the egg white preparation.

The term “principle of hydrophobic interaction” or “based on hydrophobic interaction principles” as used herein in the context of chromatography, refers to separation based on interactions between hydrophobic regions on the surface of biomolecules, e.g., proteins, and the hydrophobic surfaces of a chromatography medium.

“Reversed phase” or “reverse phase” chromatography refers to a separation technique that separates molecules according to hydrophobicity. It is thus a chromatography technique that is based on hydrophobic interactions. The reversed phase medium is highly substituted with hydrocarbon chains making it very hydrophobic. This leads to stronger interactions that, for successful elution, must be reversed using non-polar, organic solvents such as acetonitrile or methanol.

The term “hydrophobic interaction chromatography” (HIC) separates biomolecules, under relatively mild conditions by working in a more polar and less denaturing environment than reverse phase chromatograph. In HIC, hydrophobic interactions are promoted by salts so that the biomolecule, e.g., protein, is adsorbed.

### Heterologous Proteins

The methods of the invention can be used to purify any heterologous protein from egg white. For example, proteins having physical characteristics of many cytokines (i.e., small, hydrophobic, water-soluble glycoproteins that have a mass of about 8-35 kDa and an isoelectric point of between about 6.0 and about 7.0, typically, about 6.5) are purified in accordance with the invention. In certain embodiments, cytokines are purified. Examples of such cytokines include GC-SF, interferons, e.g., interferon-α and interferon-β; erythropoietin; and GM-CSF. Typically, the proteins, e.g., cytokines, purified by this procedure are usually less than about 200 amino acids in length.

A heterologous protein, e.g., cytokine, purified in accordance with the invention may be produced from a transgenic avian, such as a transgenic chicken, into which an expression construct has been introduced that provides for expression of the cytokine in the avian oviduct and deposition in an egg. The expression vector typically encodes a human cytokine. However, cytokines from other species may also be produced by an expression construct present in a transgenic avian.

### Preparation of Egg Whites

Egg whites are separated from avian eggs using known procedures. In one embodiment, the egg shell is broken and the yolk and white of the egg are poured into a container. A spoon is used to remove the yolk from the container and the process is repeated using the same container until the desired volume of egg white has accumulated.

The egg white preparation comprising the heterologous protein, e.g., cytokine, is often frozen prior to purifying the protein, although material that has not been frozen can also be used for purification. The egg white material is thawed and the protein solubilized using an acidic buffer.

The starting egg white material is acidified to a pH of less than 7.0, e.g. to a pH in the range of from about 2.0 to about 6.5. Typically, the egg white material is acidified to a pH of below 6.0, e.g. to a pH of about 4.0 to about 5.5. In some embodiment, the egg white prepared is acidified to a pH of about 5.0. Any buffer can be used to acidify the egg white preparation. The buffer for acidification may be selected so that it can be used in an ion exchange chromatography e.g., cation exchange chromatography, that is used in the purification procedure of the invention. In common embodiments, sodium acetate (NaOAc) buffers are employed.

The acidified egg white preparation may be adjusted as desired, e.g., in preparation for chromatography, by concentrating it, dialyzing it, or adding other necessary reagents. For example, the preparation is typically clarified by centrifugation or filtration.

The acidified egg white preparation is then subjected to ion exchange chromatography.

### Ion Exchange Chromatography

Ion exchange chromatography used in the methods of the invention can be either cation or anion exchange.

The anion or cation exchange resin is prepared according to known methods. Usually, an equilibration buffer is passed through the ion exchange resin prior to loading the egg white preparation comprising the protein, e.g., a cyotkine, onto the resin. Conveniently, the equilibration buffer is often the same as the loading buffer.

There are many suitable cation-exchange resins commercially available. The functional groups of cation exchange resins are typically carboxymethyl groups, sulfopropyl groups, or methyl sulfonate groups. For example, carboxymethylated, sulfonated, agarose-based, or polymeric polystyrene/divinyl benzene cation-exchange matrices can be used. Other useful matrix materials include cellulose matrices and beaded matrices; dextran, polyacrylate, polyvinyl, polystyrene, silica, and polyether matrices; and composites. Commecially available (e.g., from Amersham Biosciences, now GE Healthcare, and Sigma-Aldrich) cation exchange resins include sulfopropyl (SP) immobilized on agarose, e.g., SP-SEPHAROSE™, carboxymethylcellulose immobilized on agarose, e.g., CM SEPHAROSE, and sulphonyl immobilized on agarose, e.g., S-SEPHAROSE. Other suitable cation exchange resins for use in the invention can be readily determined by one in the art.

Suitable anion-exchange chromatography resins are also known in the art. The functional groups of anion exchange resins are typically tertiary or quaternary amino groups and include diethylaminoethyl (DEAE) groups, quaternary aminoethyl groups and quaternary ammonium groups. Matrices include agarose beads, dextran beads, polystyrene beads, and other matrices. Examples of commercially available (e.g., from Amersham Biosciences, now GE Healthcare, and Sigma-Aldrich) anion exchange resins include DEAE-SEPHAROSE, Q SEPHAROSE and others. Other suitable anion-exchange chromatography materials, as well as the selection and use of these materials for the present application, are conventional in the art.

Typically, ion exchange chromatography resins are loaded in low ionic strength buffers, e.g., in buffers having a lower salt concentration, so that the charged macromolecules, e.g., proteins, are retained by the stationary phase, which is selected to have the opposite charge. Those macromolecules, e.g., proteins, having the same charge as the stationary phase flow through without being adsorbed to the resin. The ion exchange matrix is washed with additional low ionic strength buffer to completely wash out any remaining unbound molecules, and the bound species, e.g., cytokines, are eluted using a buffer with an increased ionic strength, e.g., that has an increased concentration of salt. The elution can be performed using a gradient of increasing ionic strength, e.g., increasing salt concentration; or can be performed as a step-wise elution using one or more elution buffers that have increased ionic strength, e.g., an increased salt concentration, relative to the loading buffer.

Suitable buffers can be identified by one of skill in the art. The buffering ion should be the same charge as that of the ion exchanger. For example, phosphate and acetate buffers are often employed with cation exchange. Other common buffers, e.g. Tris buffers, may be employed in anion exchange.

An exemplary cation exchange protocol used in this invention employs two cation exchange chromatography steps. In one embodiment, one of the cation exchange chromatography steps is performed at a lower pH than the other. Thus, an acidified egg white preparation comprising a cytokine to be purified is loaded onto a cation exchange column. The preparation is loaded onto the cation exchange resin using a loading buffer that is at a pH and salt concentration such that the cytokine binds to the cation exchange resin. The column is then washed. In typical embodiments, the egg white preparation is loaded onto the column and the column is washed in a low-salt buffer having a pH of about 5.0. The cytokine-containing fraction is eluted, typically by increasing the salt concentration of the buffer relative to the salt concentration in the wash buffer.

The recovered fraction comprising the cytokine is then adjusted for a second cation exchange chromatography. Typically, the second cation exchange chromatography is performed at a different pH, usually a lower pH, i.e., a pH below 7.0. Accordingly, the pH of the cytokine-containing fraction may be adjusted to the different pH, e.g., adjusted to an acidic pH (i.e., a pH less than 7), for example, a pH less than about 5.0, e.g., a pH of about, 4.0, and loaded onto a cation exchange column. In certain embodiments, the second column is a different column that has the same resin as the first column; however, a different type of cation exchange resin may also be employed for the second chromatography. The column is washed and the cytokine-containing fraction is then eluted, typically in a buffer having an increased salt concentration relative to the loading buffer. As appreciated by those in the art, the salt concentrations of the loading and elution buffers employed in the first cation exchange chromatography can be different from the salt concentrations of the loading and elution buffers used in the second cation exchange chromatography.

Although the exemplary protocol is described in the context of cytokine purification, the protocol can also be used for the purification of other proteins, e.g., proteins that have a similar isoelectric point.

Anion exchange chromatography may also be employed, for example, and without limitation, as follows:

1. Acidified egg white preparation is adjusted to pH 7.2 with suitable buffers, e.g., tris buffer, prior to centrifugation our filtration.

2. The first anion exchange column is packed and equilibrated with a suitable buffer, e.g., tris buffer, pH 7.2.

3. The pH adjusted egg white preparation is loaded onto the first anion exchange column. The column is washed with low salt buffer and the protein, e.g., cytokine, is eluted by using an increasing salt concentration gradient.

4. The eluted cytokine fraction from the first anion exchange column is adjusted to pH 8.2.

5. The second anion exchange column is packed and equilibrated with a suitable buffer e.g., tris, pH 8.2.

6. The cytokine fraction is loaded onto the second anion exchange column and the column is washed using low salt buffer and the cytokine is eluted using an increasing salt concentration gradient.

### The cytokine-containing fraction is recovered and prepared for further chromatography using a technique based on hydrophobic interactions.

### Chromatography Based on Hydrophobic Interaction

Chromatography based on principles of hydrophobic interaction separate proteins according to their relative hydrophobicity. The resin used for separation is substituted with hydrophobic chains. Any type of chromatography based on hydrophobic interactions may be employed. These include hydrophobic interaction chromatograph and reversed phase chromatography.

Reversed phase chromatography employs a medium that is highly substituted, typically with hydrocarbon chains, to create a highly hydrophobic medium. Proteins and other macromolecules adsorb to the hydrocarbon chains in a polar buffer. The technique employs organic solvents to desorb the bound molecules.

In hydrophobic interactions chromatography, the resin typically has hydrophobic fatty acid side chains. Compared to reversed phase chromatography, the density of the ligand on the matrix is much lower. Hydrophobic interaction between a biomolecule, e.g., a protein, and the matrix is enhanced by high ionic strength buffers. The column is eluted with decreasing concentrations of salt in buffer. Proteins usually elute only when the salt concentration is very low.

In certain embodiments of the invention, reversed phase chromatography is used following ion exchange chromatography. Typically, the reversed phase chromatography is performed using high performance liquid chromatography (HPLC) or fast performance liquid chromatography (FPLC).

Both HIC and reverse phase columns are readily available commercially, e.g., from numerous vendors, e.g., Amersham Biosciences, now GE Healthcare, as well as other sources.

In one embodiment, the purification procedures described herein result in high yields of purified protein, e.g., purified cytokine.

The invention also contemplates methods of cytokine purification (e.g., purification of cytokines disclosed herein including, without limitation, erythropoietin) which do not require two ion exchange purification steps. For example, an ion exchange purification step may be used in combination with a hydrophobic interaction chromatography step and a reverse phase chromatography step.

In one specific embodiment, egg white containing a human cytokine such as erythropoietin is diluted with three volumes of 50 mM sodium acetate, pH 4.6, mixed and then filtered and loaded on to a Sepharose cation exchange column. Following a wash of the column with 50 mM sodium acetate, pH 5.0, containing 100 mM NaCl, the EPO is eluted with the acetate buffer containing 500 mM NaCl together with 0.05% Tween 20. The EPO eluted from the Sepharose column is loaded on to a Phenyl Sepharose hydrophobic interaction chromatography column. The column is equilibrated with 2 M NaCl, 50 mM Tris-HCl, pH 7.2, 0.05% Tween 20. The same buffer is used to wash the column after loading of the preparation. This is followed by a water wash. EPO is subsequently eluted with 30% IPA. This preparation is applied to a reversed-phase HPLC column and the EPO eluted with an increasing concentration of ethanol in 0.1% trifluoroacetic acid. The peak of EPO elution occurs at an ethanol concentration of about 53%. Diafiltration is used to concentrate the final EPO preparation and to replace the solvent with 0.1 M sodium phosphate buffer, pH 7.0.

Also contemplated herein are methods of purifying glycolated, e.g., pegylated, cytokines such as pegylated EPO, pegylated G-CSF and pegylated interferon, e.g., interferon α and interferon β. For example, a cytokine may purified on a cation exchange column using SP Sepharose strong cation exchange media. In one example, the buffer used for equilibration of the SP Seprarose column can be 50 mM sodium acetate, pH 4-5 diluted 80/20 in absolute ethyl alcohol, yielding a final concentration of 40 mM sodium acetate buffer and 20% ethanol. The elution buffer used for the fractionation may be composed of 50 mM sodium acetate at pH 4-5 plus 1.0 M sodium chloride (NaCl) which is diluted 80/20 in absolute ethyl alcohol to yield a final concentration of 40 mM sodium acetate and 800 mM sodium chloride in 20% ethyl alcohol (or 1.0% PEG in place of the 20% ethanol).

Prior to loading on the SP Sepharose column, the PEG-EPO pH can be adjusted to 4.5 to 5.5 by adding 50 mM sodium acetate buffer at pH=4.0±0.2. After loading, the PEG-EPO can be washed with equilibration buffer and then eluted from the column in a stepwise gradient beginning at 5% elution buffer. The elution buffer concentration is increases by steps of 5% until the monomeric form of the PEGylated EPO elutes from the column at 15% of elution buffer. The unPEGylated EPO is eluted at 100% of elution buffer.

Following purification, PEG-EPO is buffer exchanged using stir cell technology well know to those of ordinary skill in the art. The molecular weight cut off of the stir cell is 10,000 daltons. The resulting product can be buffer exchanged to pharmaceutical formulation containing 0.05 mg polysorbate 80, 2.12 mg sodium phosphate monobasic, 0.66 mg sodium phosphate dibasic anhydrous, and 8.18 mg sodium chloride per ml with a pH of 6.2±0.2.

It will be apparent to those skilled in the art that various modifications, combinations, additions, deletions and variations can be made in the present invention without departing from the scope or spirit of the invention. For instance, features illustrated or described as part of one embodiment can be used in another embodiment to yield a still further embodiment. It is intended that the present invention covers such modifications, combinations, additions, deletions and variations as come within the scope of the appended claims and their equivalents.

The present invention is further illustrated by the following examples, which are provided by way of illustration and should not be construed as limiting. The contents of all references, published patents and patents cited throughout the present application are hereby incorporated by reference in their entireties.

### EXAMPLE 1

### Purification of GCSF from Egg White.

### GCSF-Containing Egg White Preparation

For batch 1, frozen egg white (29.6 kg) comprising heterologous GCSF was thawed at 2-8° C. The thawed egg white was transferred to a 100-liter container. A volume of 88.8 liters (3 volumes of egg white) of 50 mM NaOAc, pH 4.6 was added into the thawed egg white for a total volume of about 126.8 liters. The pH of the mix was pH 5.0+0.1. The diluted egg white mix was stirred at 2-8° C. overnight at 150 RPM.

After overnight-mixing, the preparation was centrifuged at 9,000×g for 15 minutes. (Large scale filters may also be employed for clarification of the egg white material). The clarified supernatant was transferred to a 30-liter container and filtered into 20-liter storage bags with a 0.22μm filter.

### Chromatography Purification of GCSF with a SP1 Column

A chromatography column was assembled following the user's manual. The column size was 30 (ID)×29 (H) cm (20 liters). The chromatography resin was SP Sepharose Fast Flow resin (cation exchange). The column flow rate was adjusted to 1.6 liters/min (linear flow rate was at about 135 cm/hr). After the assembly of the column, the column was washed with 20 liters of 1 N NaOH, 40 liters of 2 M NaCl, and 60 liters of Nanopure water. The column was then equilibrated with 60 liters of 50 mM NaOAc, pH 5.0.

The total volume of 126.8 liters of processed GCSF-containing egg white prepared as described above was then loaded onto the SP1 column at 1.6 liters/min. An on-line 0.22μm filter was used prior to loading egg white to the SP1 column. After completion of loading, the SP1 column was washed with 60 liters of 50 mM NaOAc, pH 5.0 and 60 liters of 50 mM NaOAc/20 mM NaCl, pH 5.0.

The GCSF-containing fraction was eluted with 80 liters of 50 mM NaOAc/50 mM NaCl, pH 5.0. The eluted GCSF fraction was adjusted to pH 4.0 with 1 N HCl (36.5 ml of 1 N HCl added/L of eluted fraction). Tween 80 was then added to the GCSF fraction to a final concentration of 0.001%. The processed GCSF fraction was then stored at 2-8° C. for further processing.

The purification yield and recovery from the first step chromatograph steps is shown in below Table 1:

| | | Elution | Elution | Elution |
|---|---|---|---|---|
| | Preload | Bag# 1 | Bag# 2 | Bag# 3 |
| Volume (L) | 126.8 | 15.5 | 57 | 10.4 |
| GCSF (mg/L) | 0.98 | 0.42 | 1.6 | 0.44 |
| GCSF (mg) | 124.3 | 6.5 | 91.2 | 4.6 |

| | | |
|---|---|---|
| Total GCSF (mg) | 124.3 | 102.3 |
| Recovery | | 82% |

A second batch was prepared in the same manner as described for the first batch. The Elution Bag#2 from each of the two batches was combined for further processing as described below.

### Chromatography Purification of GCSF with a SP2 Column

A chromatography column of 14 (ID)×29 (H) cm (4.5 liters) was assembled. The chromatography resin was SP Sepharose Fast Flow (cation exchange). The column flow rate was adjusted to 380 ml/min (linear flow rate is at 150 cm/hr).

After the assembly of the column, the column was washed with 4.5 liters of 1 N NaOH, 9 liters of 2 M NaCl, and 13.5 liters of WFI water. The column was then equilibrated with 13.5 liters of 50 mM NaOAc, pH 4.0.

The G-CSF-containing fractions from the two SP1 batches were loaded onto the SP2 column at 380 ml/min. After completion of loading, the SP2 column was washed with 22.5 liters of 50 mM NaOAc/150 mM NaCl, pH 4.0. The GCSF fraction was eluted with 18 liters of 50 mM NaOAc/200 mM NaCl, pH 4.0. Tween 80 was then added to the GCSF fraction to a final concentration of 0.001%. The processed GCSF fraction was then stored at 2-8° C. for further processing. The purification yield and recovery following this step are shown below in Table 2:

| | Preload | |
|---|---|---|
| | Batch 1 + 2 | Elution |
| Volume (L) | 114 | 14 |
| GCSF (mg/L) | 1.64 | 8.4 |
| GCSF (mg) | 187 | 117.6 |
| Recovery | | 63% |

### Chromatography Purification of GCSF with Reverse Phase HPLC

A Vydac C4 reverse phase chromatography column was then employed to further purify the GCSF. The column was attached to a Waters HPLC system. The column size was 2.2×25 cm (95 ml). The column flow rate was adjusted to 22 ml/min (linear flow rate was at 360 cm/hr). After the column was connected to the HPLC system, the column was washed with 500 ml of 90% IPA/0.1% TFA and then equilibrated with 1 liter of 5% IPA/0.1% TFA.

Prior to loading the GCSF fractions, eluted from the SP2 column) onto the reverse phase column, IPA was added to the preparation to adjust to a final concentration of 5%. The GCSF fraction (14 liters) eluted from SP2 column was loaded onto the reverse phase HPLC column at the flow rate of 22 ml/min. After completion of loading, the column was washed with 300 ml of 5% IPA/0.1% TFA. The GCSF was eluted from the column with a gradient elution method. The elution was programmed as the follows:

| | | |
|---|---|---|
| 0-110 | ml | 0% B |
| 110-220 | ml | 0-40% B |
| 220-770 | ml | 40-60% B |
| 770-1760 | ml | 60-100% B |
| 1760-1870 | ml | 100% B |
| 1870-2090 | ml | 0% B |

Buffer A: 5% IPA/0.1% TFA
Buffer B: 90% IPA/0.1% TFA

The volume of 690 ml of GCSF peak was collected and held for 30 minutes for virus inactivation. A volume of 6.21 L of 50 mM NaOAc (pH 5)/0.001% Tween 80 was then added into the GCSF solution for neutralization. The purified GCSF was stored at 2-8° C. for further processing. The purification yield and recovery are shown below in Table 3:

| | Preload | Elution |
|---|---|---|
| Volume (L) | 13 | 6.9 |
| GCSF (mg/L) | 16.6 | 31.4 |
| GCSF (mg) | 216 | 217 |
| Recovery | | 100% |

### EXAMPLE 2

### Purification of Interferon-α from Egg White.

### IFN-containing Egg White Preparation

For batch 1, frozen egg white (40 kg) containing interferon-α was thawed at 2-8° C. The thawed egg white was transferred to a 100-liter container. A volume of 120 liters (3 volumes of egg white) of 50 mM NaOAc, pH 4.6 was added to the thawed egg white. The pH of the mix was about pH 5.0+0.1. The diluted egg white mix was stirred at 2-8° C. overnight at 150 RPM.

After overnight-mixing, the egg white preparation comprising the interferon protein was centrifuged at 9,000×g for 15 minutes. (Large scale filters can alternatively be used for clarification). The clarified supernatant was transferred to a 30-liter container and filtered into 20-liter storage bags with 0.22μm filters.

Chromatography Purification of IFN with a SP1 Column A 20-liter chromatography column (30×29 cm) was assembled following the user manual. The chromatography resin was SP Sepharose Fast Flow resin (cation exchange). The column flow rate was adjusted to 1.6 liters/min (linear flow rate is at 150 cm/hr). After the assembly of the column, the column was washed with 20 liters of 1 N NaOH, 40 liters of 2 M NaCl, and 60 liters of Nanopure water. The column was then equilibrated with 60 liters of 50 mM NaOAc, pH 5.0.

The total volume of about 160 liters of processed IFN-containing egg white prepared as described above was loaded onto the SP1 column at 1.6 liters/min. An on-line 0.22μm filter was used prior to loading egg white onto the SP1 column. After completion of loading, the SP1 column was washed with 105 liters of 50 mM NaOAc/50 mM NaCl, pH 5.0. The IFN fraction was eluted with 84 liters of 50 mM NaOAc/100 mM NaCl, pH 5.0. The eluted IFN fraction was then adjusted to pH 4.0 with 1 N HCl (36.5 ml of 1 N HCl added/L of eluted fraction). The processed IFN fraction was stored at 2-8° C. for further processing.

The purification yield and recovery from this chromatography step is shown below in Table 4:

| | | Elution | Elution | Elution |
|---|---|---|---|---|
| | Preload | Bag# 1 | Bag# 2 | Bag# 3 |
| Volume (L) | 160 | 19.7 | 54.9 | 12.4 |
| IFN (mg/L) | 1.4 | 0.1 | 3.7 | 0.2 |
| IFN (mg) | 217 | 2 | 203 | 2.5 |

| | | |
|---|---|---|
| Total IFN (mg) | 217 | 207.5 |
| Recovery | | 96% |

A second batch was prepared in the same manner as described for the first batch. The Elution Bag#2 from each of the two batchs was combined for further processing described below.

### Chromatography Purification of IFN with a SP2 Column

A 4-liter chromatography column (14×26 cm) was assembled following the user manual. The chromatography resin was SP Sepharose Fast Flow resin. The column flow rate was adjusted to 380 ml/min (linear flow rate is at 150 cm/hr). After assembly of the column, the column was washed with 4 liters of 1 N NaOH, 8 liters of 2 M NaCl, and 12 liters of WFI water; and then equilibrated with 12 liters of 50 mM NaOAc, pH 4.0.

The combined IFN-containing fractions from the two SP1 batches were loaded onto the SP2 column at 380 ml/min. After completion of loading, the SP2 column was washed with 20 liters of 50 mM NaOAc/150 mM NaCl, pH 4.0. The IFN fraction was eluted with 16 liters of 50 mM NaOAc/360 mM NaCl, pH 4.0 and stored at 2-8° C. for further processing.

The purification yield and recovery from this chromatography are shown below in Table 5:

| | Preload | Elution | Elution | Elution |
|---|---|---|---|---|
| | Lot 1 | Bag# 1 | Bag# 2 | Bag# 3 |
| Volume (L) | 110 | 2.5 | 11 | 2.5 |
| IFN (mg/L) | 4.4 | 3 | 17 | 2.6 |
| IFN (mg) | 484 | 7.5 | 187 | 6.5 |

| | | |
|---|---|---|
| Total IFN (mg) | 484 | 201 |
| Recovery | | 41% |

### Chromatography Purification of IFN with Reverse Phase HPLC (Vydac C4 Column)

A Vydac C4 reverse phase chromatography column was attached to a Waters HPLC system. The column size was 5×25 cm (10μm, 490 ml). The column flow rate was adjusted to 75 ml/min (linear flow rate is at 230 cm/hr). After the column was connected to HPLC system, the column was washed with 500 ml of 99.9% Acetonitrile/0.1% TFA and then equilibrated with 1 liter of 5% Acetonitrile/0.1% TFA.

The IFN fractions (11 liters) eluted from SP2 column was loaded onto the reverse phase HPLC column at a flow rate of 75 ml/min. After completion of loading, the column was washed with 500 ml of 5% Acetonitrile/0.1% TFA. The IFN was eluted from the column with a gradient elution method. The gradient was programmed as the follows:

| | | |
|---|---|---|
| 0-500 | ml | 0% B |
| 500-1500 | ml | 0-35% B |
| 1500-3000 | ml | 35% B |
| 3000-5500 | ml | 35-55% B |
| 5500-6500 | ml | 55% B |
| 6500-7500 | ml | 0% B |

Buffer A: 5% Acetonitrile/0.1% TFA
Buffer B: 99.9% Acetonitrile/0.1% TFA

The volume of 1.8 L of IFN peak was collected and held for 30 minutes for virus inactivation. A volume of 1.46 L of 50 mM Tris/0.01% Tween 80, pH 5, was then added into the IFN solution for neutralization and the purified IFN was stored at 2-8° C. for further processing.

The purification yield and recovery are shown below in Table 6:

| | Preload | Elution |
|---|---|---|
| Volume (L) | 11 | 3.26 |
| IFN (mg/L) | 10.3 | 18.9 |
| IFN (mg) | 113 | 61.6 |
| Recovery | | 54.5% |

### Chromatography Purification of IFN with Reverse Phase HPLC (Diazem Peptica C4-A Column)

A Diazem Peptica C4-A reverse phase chromatography column is attached to Waters HPLC system. The column size was 2.2×25 cm (5μm, 95 ml). The column flow rate was adjusted to 20 ml/min (linear flow rate is at 316 cm/hr). The column was washed with 200 ml of 99.9% Acetonitrile/0.1% TFA and equilibrated with 300 ml of 5% Acetonitrile/0.1% TFA.

The IFN fractions eluted from the first reverse phase HPLC column (Vydac Protein C4) was loaded onto the reverse phase HPLC column (Diazem Peptica C4-A) at a flow rate of 20 ml/min. After completion of loading, the column was washed with 500 ml of 30% Acetonitrile/0.1% TFA and the IFN was eluted with a gradient elution method. The gradient was programmed as the follows:

| | | |
|---|---|---|
| 0-500 | ml | 35% B |
| 500-1400 | ml | 35-45% B |
| 1400-1600 | ml | 100% B |

Buffer A: 5% Acetonitrile/0.1% TFA
Buffer B: 99.9% Acetonitrile/0.1% TFA

The volume of 514 ml of IFN peak was collected. A volume of 4.6 L of 50 mM Tris/0.01% Tween 80, pH 5, was then added to the IFN solution for neutralization and the purified IFN was stored at 2-8° C.

The purification yield and recovery from this chromatography are shown in Table 7:

| | Preload | Elution |
|---|---|---|
| Volume (L) | 3.9 | 5.1 |
| IFN (mg/L) | 18.9 | 12.6 |
| IFN (mg) | 74.8 | 64.3 |
| Recovery | | 86% |

All publications, patents, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of purifying a heterologous cytokine that is expressed in egg whites, the method comprising:
acidifying an egg white preparation comprising the heterologous cytokine;
performing a first and a second ion exchange chromatography; and
performing chromatography based on hydrophobic interaction, thereby purifying the heterologous cytokine.

2. The method of claim 1, wherein the heterologous cytokine is granulocyte colony-stimulating factor.

3. The method of claim 1, wherein the heterologous cytokine is interferon-α.

4. The method of claim 1, wherein the heterologous cytokine is erythropoietin.

5. The method of claim 1, wherein the first and the second ion exchange chromatographies are cation exchange chromatography.

6. The method of claim 5, wherein the second cation exchange chromatography is at a different pH than the first cation exchange chromatography.

7. The method of claim 1, wherein the chromatography based on a hydrophobic interaction is a reversed phase chromatography.

8. The method of claim 1, wherein the chromatography based on a hydrophobic interaction is a hydrophobic interaction chromatography.

9. The method of claim 1, wherein the egg white preparation is acidified to a pH in the range of about 2.0 to about 6.5.

10. The method of claim 9, wherein the egg white preparation is acidified to a pH in the range of about 4.0 to about 6.0.

11. The method of claim 10, wherein the egg white preparation is acidified to a pH of about 5.0.

12. A method of purifying a heterologous protein from an egg white preparation comprising the heterologous protein, the method comprising:
acidifying the egg white preparation to a pH of about 4.0 to about 6.0;
performing a first cation exchange chromatography;
performing a second cation exchange chromatography, wherein the second cation exchange chromatography is at a lower pH than the first cation exchange chromatography; and
performing chromatography based on hydrophobic interaction; thereby purifying the heterologous protein from the egg white preparation.

13. The method of claim 12, wherein the heterologous protein is a cytokine.

14. The method of claim 12, wherein the heterologous protein is selected from the group consisting of granulocyte colony-stimulating factor (GCSF), GM-CSF, interferon-α, interferon-β, and erythropoietin.

15. The method of claim 12, wherein the chromatography based on hydrophobic interaction is a reversed phase chromatography.

16. The method of claim 12, wherein the chromatography based on hydrophobic interaction is a hydrophobic interaction chromatography.

17. The method of claim 12, wherein the first cation exchange chromatography is at a pH of about 5.0 and the second cation exchange chromatography is at a pH of about 4.0.

18. The method of claim 17, further wherein the hydrophobic interaction chromatography is a reversed phase chromatography.

19. The method of claim 18, further wherein the heterologous protein is granulocyte colony-stimulating factor (GCSF).

20. The method of claim 18, further wherein the heterologous protein is interferon-α.

21. The method of claim 18, further wherein the heterologous protein is erythropoietin.

22. The method of claim 18, further wherein the heterologous protein is GM-CSF.

23. The method of claim 18, further wherein the heterologous protein is interferon-β.
